(19) United States
(12) Patent Application Publication
(10) Pub. No.: US 2006/0121463 A1
(43) Pub. Date: Jun. 8, 2006

(54) Allelic variation in human gene expression

(75) Inventors: Kenneth Kinzler, Baltimore, MD (US); Bert Vogelstein, Baltimore, MD (US); Hai Yan, Chapel Hill, NC (US)

Correspondence Address: BANNER & WITCOFF, 1001 G STREET N W, SUITE 1100, WASHINGTON, DC, 20001, US

(73) Assignee: The John Hopkins University, Baltimore, MD (US)

(21) Appl. No.: 10/516,492

(22) PCT Filed: Jun. 4, 2003
(86) PCT No.: PCT/US03/17262

§ 371 Date: Jan. 27, 2006

Related U.S. Application Data
(60) Provisional application No. 60/385,901, filed on Jun. 6, 2002.

(51) Int. Cl. C12Q 1/68

(52) U.S. Cl. 435/6

(57) ABSTRACT

Genetically-determined variation in expression levels is an important component of human diversity and has significant implications for normal and abnormal human physiology. Using this genetically determined variation one can identify disease risk factors in individuals. One can associate such variations with birth defects, diseases, and non-disease traits. Such variations can be associated with susceptibility or resistance to the effects of drugs or other therapeutic interventions.

The U.S. government retains certain rights in the invention by virtue of its support of the underlying work involved in making the invention, and the terms of grants from the National Institutes of Health grants CA57345, CA 62924 and CA43460.

### BACKGROUND OF THE INVENTION

1. Field of the Invention

The invention relates to the field of diagnostic and prognostic testing. In particular it relates to detecting variations in gene expression between individuals in a population that may indicate disease susceptibility or predict the phenotype of traits deemed within normal variation.

2. Background of the Prior Art

Understanding the genetic basis of human variation is one of the most important goals of modem biomedical research. Much work in this area is focused on genetic polymorphisms associated with structural alterations of the encoded proteins. However, studies in other organisms suggest that such protein polymorphisms account for only a fraction of normal variation and that differences in gene expression levels account for a major part of the variation within and among species (1, 2). In humans, altered gene expression has not been systematically addressed in the context of normal human variation.

There is a need in the art for techniques for assessing variation in gene expression and for associating such variations with disease states and disease susceptibility.

### BRIEF SUMMARY OF THE INVENTION

In a first embodiment of the invention a method of associating a genotype with a phenotype is provided. Levels of expression of an allele of a gene in a first population comprising affected individuals are determined. The affected individuals share a phenotype. Levels of expression of the allele in a second population comprising control individuals are determined. The control individuals do not share the phenotype. The levels of expression of the allele in the first and the second populations are compared. An allele whose expression differs in a statistically significant manner between the first and the second populations is identified as having an association with the phenotype.

In a second embodiment of the invention a method is provided for measuring allelic expression variation in a non-imprinted gene in an individual. Messenger RNA (mRNA) from an individual heterozygous for a single nucleotide polymorphism (SNP) in a non-imprinted gene is reverse transcribed and amplified to form first cDNA from a first allele and second cDNA from a second allele. Primers are hybridized to the first cDNA and the second cDNA. Those primers hybridized to the first cDNA and the second cDNA are differentially labeled to form differentially labeled first and second primers. The amount of differentially labeled first primers is compared to the amount of differentially labeled second primers. A statistically significant difference between the amount of labeled first primers and the amount of labeled second primers indicates that the first and second alleles are differentially expressed in the first individual.

In a third embodiment, a method is provided for measuring allelic expression variation in a non-imprinted gene in an individual. Messenger RNA (mRNA) from an individual heterozygous for a single nucleotide polymorphism (SNP) in a non-imprinted gene is reverse transcribed and amplified to form first cDNA from a first allele and second cDNA from a second allele. Primers are hybridized to first cDNA and second cDNA. Those primers hybridized to the first cDNA are differentially labeled from those hybridized to the second cDNA using fluorescent dye terminators and a single base extension reaction to form differentially labeled first and second primers. The amount of differentially labeled first primers is compared to the amount of differentially labeled second primers using capillary electrophoresis. A statistically significant difference in the amount of labeled first primers from the amount of labeled second primers indicates that the first and second alleles are differentially expressed in the individual.

In a fourth embodiment of the invention a method is provided for measuring allelic expression variation in a non-imprinted gene in a first individual. Level of expression of an allele of a gene in a first individual displaying a phenotype is determined, as is the level of expression of the allele in a population of control individuals. The control individuals do not display the phenotype. Level of expression of the allele in the first individual is compared to level of expression in the population of control individuals. A statistically significant difference in the levels of expression indicates that the allele in the first individual may be associated with the phenotype.

These and other embodiments of the invention provide the art with an additional dimension for assessing genetic diversity.

### BRIEF DESCRIPTION OF THE DRAWINGS

FIG. 1 shows a schematic of assay for fractional allelic expression showing key steps. See text for additional details.

FIG. 2 shows the result of allelic expression analyses performed as described below in note (3). Representative results are shown for eight genes. The shaded box represents approximated 95% confidence interval and red bars indicate individuals displaying significant variations, as defined in note (6).

FIG. 3 shows examples of two kindreds exhibiting Mendelian inheritance patterns in either the PKD2 or Calpain-10 gene. Only individuals who were heterozygous for the SNP or were used to deduce haplotypes are shown. The individuals displaying altered fractional allelic expression are shaded red, and the individuals originally found to display altered expression are indicated by arrows. An obligate carrier in the PKD2 pedigree who could not be scored is indicated with a red dot. The results of genotype analyses are shown directly above each member of the pedigrees. The markers employed are listed at the right and each allele observed in a family was assigned a number. Markers suggesting a recombination are underlined and the allele associated with altered expression is indicated in red. The fractional allelic expression data used to score the pedigree are shown above the genotype and were interpreted as described in the legend to FIG. 2.

### DETAILED DESCRIPTION OF THE INVENTION

We have here developed methods to quantitatively evaluate allelic variation in gene expression and applied them to the analysis of 13 different genes. We found allelic variation in expression levels in six of these genes, and showed that these variations were often heritable. The results suggest that genetically-determined variation in expression levels is an important component of human diversity and have significant implications for normal and abnormal human physiology.

Phenotypes which can be assessed according to the present invention are those which relate to disease as well as those which relate to normal human physiology. Examples of phenotypes include disease susceptibility, birth defects, psychological parameters, learning parameters, and physical characteristics. The phenotype is preferably a polymorphic phenotype, ie., many forms of the characteristic exist. Individuals who share a particular phenotype are grouped together and are termed “affected individuals” for purposes of this invention. Individuals who do not share the particular phenotype are used to form a control population.

Levels of expression of an allele can be determined using any techniques which are known in the art. Such techniques include but are not limited to allele-specific expression assays, oligonucleotide ligase assays, and dideoxy single-base extension of an unlabeled oligonucleotide primer, described in more detail below. Any technique can be used that can distinguish between expression products of alleles. The level of expression of a single allele of a gene can be determined in isolation, without comparing expression to the second allele present in an individual. Alternatively, the level of expression of one allele of a gene in an individual can be compared to the level of a second allele of the gene in the individual.

Levels of expression are compared to determine statistically significant differences. Any statistical analysis can be used which determines such differences. One particular analysis which can be used is the MIED procedure of the SAS system version 8.0 for repeated measurements. A statistically significant difference can be a 5% difference, a 10% difference, a 15% difference, a 20% difference, a 25% difference, or more.

Haplotypes that are associated with an altered level of expression of an allele can be determined. The haplotypes can be used as surrogates for the altered level of expression. The haplotypes can be used to follow the altered expression levels either within a population or within a family.

Variations in expression can be determined to be heritable if they are determined in related individuals, such as parents and offspring. If the variation in expression is determined to be consistently inherited along with at least two adjacent microsatellite markers, for example, then the variation is indicated to be heritable.

A heritable variation in expression levels can be studied to determine any changes in sequence which might account for the expression alteration. Such changes are likely to be located in control regions such as the promoter, although they can occur elsewhere. The changes can be subtle, single base pair changes or they can be insertions or deletions. Such changes can be determined by mapping and/or sequencing or other techniques known in the art for determining genetic changes.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### EXAMPLE

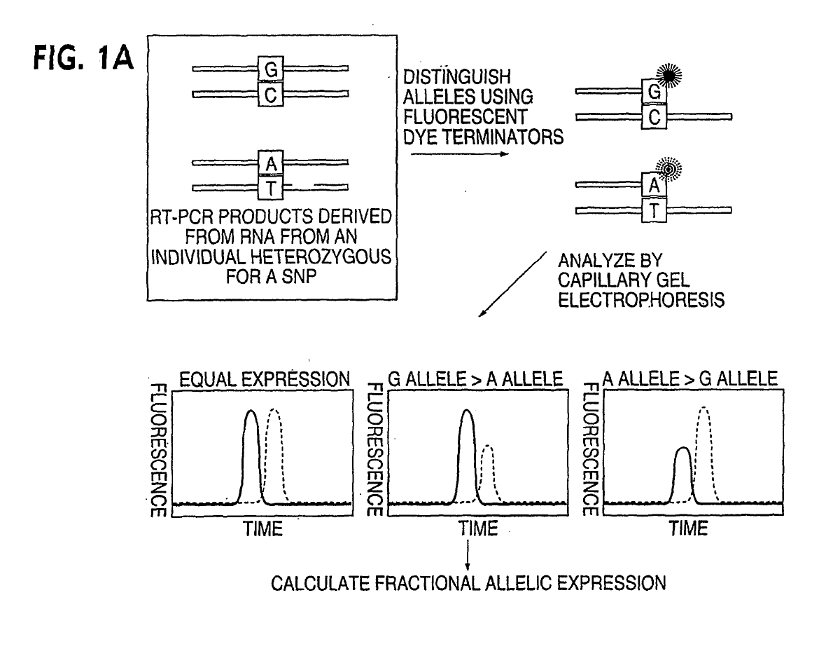

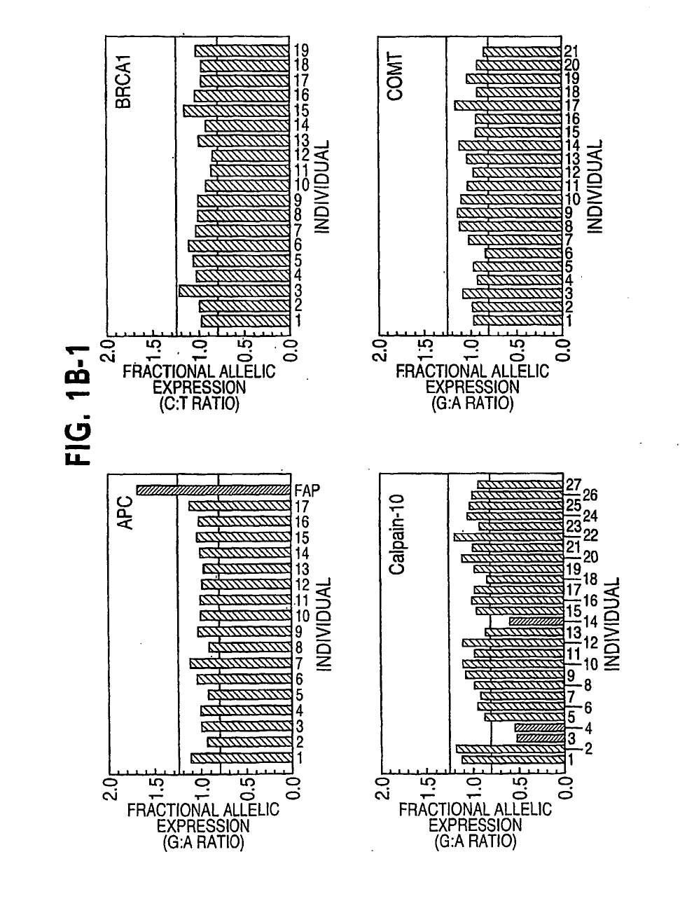

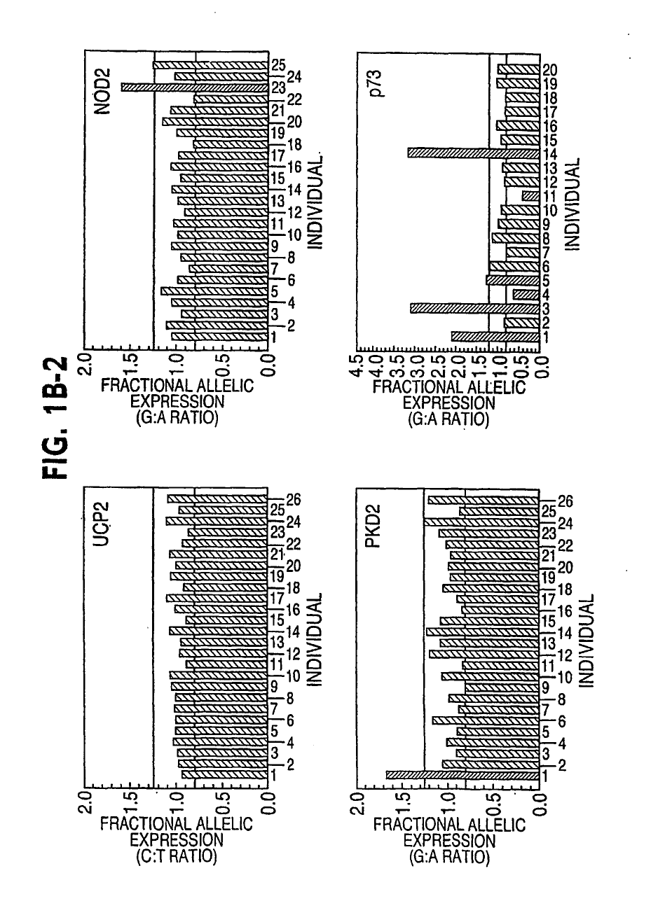

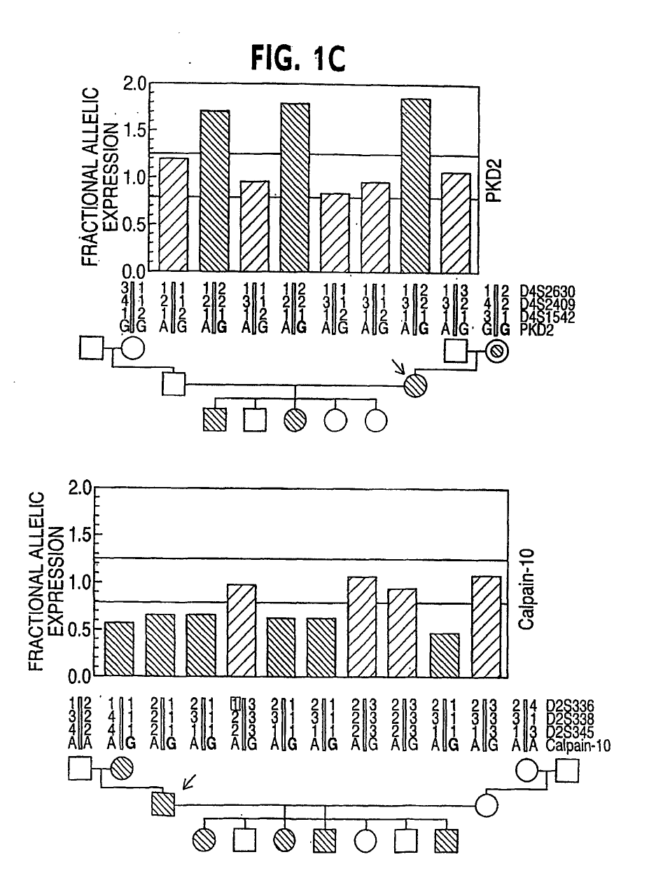

The analysis of variation of gene expression is complicated by the expected magnitude of the differences; complete loss of expression from one allele results in a reduction of total expression levels of only 50%. However, comparing expression of one allele to the other can greatly facilitate the detection of such differences. Importantly, such comparisons ensure that the alleles are both expressed within the identical intracellular environment and are independent of environmental factors. To make these comparisons, we studied RT-PCR products derived from the mRNA of normal individuals who were heterozygous for SNPs within the studied transcripts (FIG. 1A). The PCR products derived from each allele were then distinguished using differentially labeled fluorescent dideoxy terminators in single nucleotide extensions. The products were quantified by capillary gel electrophoresis and reproducibility was ensured by the analysis of seven replicates of each sample (FIG. 1A).

We applied this approach to lymphoblastoid cells derived from 96 normal individuals from CEPH reference families (3). To validate our approach, we first examined allelic expression of the APC tumor suppressor gene (APC) in CEPH individuals and in an FAP patient previously shown to have decreased expression of one allele (4). No significant variation in fractional allelic expression was observed in any of 17 heterozygous CEPH individuals tested (5). In contrast, unequal allelic expression was detectable in the FAP patient (FIG. 1B). Based on these and other control analyses, we estimate that we were able to confidently identify variation when the differences between expression of the two alleles differed by more than 20% (6).

We next examined variation in 12 additional genes containing relatively common SNPs (Table 1). For each gene, we first studied genomic DNA to determine which of the 96 individuals were heterozygous at these loci, and identified on average 23 heterozygous individuals for further study. Significant differences in allelic variation were observed in 6 of these 12 genes. The fraction of patients exhibiting variation in allelic expression ranged from 3% (one of 37 individuals tested for Catalase) to 30% (six of 20 individuals tested for p73) (Table 1 and FIG. 1B). In those individuals whose alleles were differentially expressed, the ratio of transcripts varied from 1.3:1 (FBNI to 4.3:1 (p73).

Given that these variations were each observed in a minority of individuals, it is unlikely they were due to genetic imprinting. It was not possible to determine if the altered expression was due to increased or decreased expression of the rare allele from these analyses.

To determine whether the variations were heritable, we examined the families of nine individuals exhibiting allelic variation in the assays described above. Six of these families proved uninformative (7). The other three families were informative and each displayed a pattern of expression fully consistent with Mendelian inheritance. These included two families with allelic variation of Calpain-10 expression and one family with allelic variation of PKD2 expression (examples in FIG. 1C). In each of the families, the altered expression was found to be consistently inherited with a single haplotype defined by at least two adjacent microsatellite markers. Moreover, it was possible to deduce the nature of the altered allelic expression from these family studies. In the case of PKD2, the altered allelic expression was due to increased expression of the affected allele whereas in both Calpain-10 families, it was due to decreased expression of the affected allele.

These findings provide strong evidence that cis-acting, inherited variations in gene expression are relatively common among normal individuals. In this regard, it is important to note that our measurements likely represent an underestimate of such differences in gene expression as they were derived from a single cell type and additional variations in allelic expression may manifest in a cell-type specific manner.

While we have focused on normal differences in allelic expression in this study, our results have obvious implications for disease susceptibility. They suggest an approach for connecting genotype to phenotype in which the expression levels of genes are measured in patients and compared to controls. This strategy would have two clear advantages over methods based on linkage as commonly used in association, sib-pair, and related studies (8,9). First, any expression differences noted would provide direct evidence for the implicated gene's causal role, while linkage data can at best implicate that some gene in the linked region is responsible for the phenotype. Second, expression data are independent of population structure and do not rely on the absence of recombination between the marker and the responsible gene. We anticipate that the approach described above or other methods for measuring allelic variation in gene expression will play a major role in defining normal human variation and disease susceptibility in the future.

TABLE 1
Allelic Variation in Gene Expression

| | | Heterozygous | Individuals | |
|---|---|---|---|---|
| | | Individuals | Displaying | Magnitude of |
| Gene | SNP | Tested | Variations | Variation (fold) |
| APC | C486T | 17 | 0 | — |
| BRCA1 | T4449C | 19 | 0 | — |
| Calpain-10 | A2037G | 27 | 3 (11%) | 1.7-7.9 |
| Catalase | T1235C | 37 | 1 (3%) | 1.4 |
| COMT | C388T | 21 | 0 | — |
| DNT | A195G | 20 | 0 | — |
| FBN1 | T2008C | 19 | 2 (11%) | 1.3, 1.6 |
| LDLR | G2325A | 24 | 0 | — |
| NOD2 | T1866G | 25 | 1 (4%) | 1.6 |
| p53 | G466C | 18 | 0 | — |
| p73 | T629C | 20 | 6 (30%) | 1.5-4.3 |
| PKD2 | G4208A | 26 | 1 (4%) | 1.7 |
| UCP2 | C544T | 26 | 0 | — |

### NOTES AND REFERENCES

1. N. A. Johnson, A. H. Porter, J Theor Biol 205, 52742. (2000).
2. M. Levine, Nature 415, 848-9 (Feb. 21, 2002).
3. Lymphoblastoid cell lines representing two genetically unrelated individuals from each of 48 CEPH references families were obtained from the National Institute of General Medical Sciences repository maintained by the Coriell Institute for Medical Research. Cells were grown in RPMI with 10% FBS, and mRNA was isolated from 2×10₆ cells using Amersham Pharmacia QuickPrep micro mRNA purification kit. RT-PCR products from each allele of the gene of interest were distinguished using ABI Prism SNaPshot Multiplex Kit and analyzed on a SpectruMedix SCE9610 Genetic Analysis system. Sequences of the primers used for PCR amplification and SNP determination are available upon request.
4. H. Yan et al.,
Nat Genet 30, 256 (January 2002).
5. The fractional allelic experiment for each sample was determined through seven replicates. Prior to subsequent statistical analyses, obvious technical failures or statistical outliers were eliminated. In no case did this result in elimination of more than three replicates and on average resulted in elimination of one in every 25 data points. The data were then analyzed using the MIXED procedure of the SAS system version 8.0 for repeated measurements. This analysis revealed that none of the 17 individuals tested for expression of APC had a fractional allelic expression value that exceeded the 95% confidence interval for the mean. In contrast, the control FAP patient was well outside these limits.
6. Analysis of the APC allelic expression ratios of normal individuals using the MIXED procedure of the SAS system version 8.0 yielded 95% confidence intervals ranging from 0.79 to 1.27 (average 0.82 to 1.22). Because no significant variation in expression of APC could be detected in these 17 individuals or in 24 individuals by a digital-PCR based approach (4), we concluded that there was little genetic variation in APC expression and could thereby be used to model our analysis of other genes where the extent of variation was unknown. For these genes, samples initially falling outside the 95% confidence interval described above were evaluated through additional experiments. We required that any differences interpreted to represent variations in allelic expression be observed in multiple independent RNA samples and where possible, confirmed with an antisense primer.
7. Six families were deemed not informative. In five of these families, the spouse of the individual exhibiting an altered allelic expression ratio was homozygous for the SNP. In one family showing variations in FBNI expression, altered allelic expression was detected in individuals from both the maternal and paternal sides of the pedigree, precluding unequivocal assignment of expression status in the offspring.
8. P. O. Brown, L. Hartwell, Nat Genet 18, 91-3 (February 1998).
9. J. Ott, J. Hoh, Am J Hum Genet 67, 289-94 (August 2000).

1. A method of associating a genotype with a phenotype, comprising:
determining levels of expression of an allele of a gene in a first population comprising affected individuals, said affected individuals sharing a phenotype;
determining levels of expression of the allele in a second population comprising control individuals, said control individuals not sharing the phenotype;
comparing levels of expression of the allele in the first and the second populations;
identifying an allele whose expression differs in a statistically significant manner between the first and the second populations as having an association with the phenotype.

2. The method of claim 1 wherein the phenotype is a disease susceptibility.

3. The method of claim 1 wherein the phenotype is a disease.

4. The method of claim 1 wherein the phenotype is a birth defect.

5. The method of claim 1 wherein the affected individuals are heterozygous for the gene.

6. The method of claim 1 wherein the control individuals are heterozygous for the gene.

7. The method of claim 1 wherein the phenotype is a polymorphic phenotype.

8. The method of claim 1 wherein expression of the allele is determined independent of the expression of other alleles of the gene.

9. The method of claim 1 wherein the phenotype is not related to a known disease.

10. The method of claim 1 further comprising determining a haplotype associated with the allele in the first population.

11. The method of claim 1 wherein the level of expression of the allele is heritable.

12. The method of claim 1 further comprising determining a sequence variation which is associated with the allele in the first population.

13. The method of claim 12 wherein the sequence variation is a single nucleotide polymorphism (SNP).

14. The method of claim 12 wherein the sequence variation is an insertion.

15. The method of claim 12 wherein the sequence variation is a deletion.

16. The method of claim 12 further comprising determining that the sequence variation causes the level of expression of the allele to differ from level of expression of at least one other allele of the gene.

17. The method of claim 1 wherein the levels of expression are determined and compared using fluorescent dye terminators and a single-base extension reaction.

18. The method of claim 17 wherein the levels of expression are determined and compared using capillary electrophoresis.

19. A method of measuring allelic expression variation in a non-imprinted gene in a first individual, comprising:
reverse transcribing and amplifying mRNA from an individual heterozygous for a single nucleotide polymorphism (SNP) in a non-imprinted gene to form first cDNA from a first allele and second cDNA from a second allele;
hybridizing primers to first cDNA and second cDNA and differentially labeling those primers hybridized to first cDNA and second cDNA to form differentially labeled first and second primers;
comparing amount of differentially labeled first primers to amount of differentially labeled second primers, wherein a statistically significant difference in the amount of labeled first primers from the amount of labeled second primers indicates that the first and second alleles are differentially expressed in the first individual.

20. The method of claim 19 wherein the differential labeling is performed using fluorescent dye terminators.

21. The method of claim 19 wherein the comparing is performed using capillary electrophoresis.

22. The method of claim 19 wherein the differential labeling is performed using a single base extension reaction.

23. The method of claim 19 further comprising measuring allelic expression of the first or second allele in a second individual related to the first individual to confirm that the allelic expression variation is heritable.

24. The method of claim 23 wherein the second individual is a parent or offspring of the first individual.

25. The method of claim 23 wherein the first and second alleles are both expressed in the first individual.

26. The method of claim 19 wherein the statistically significant difference is at least 20%.

27. The method of claim 19 further comprising determining a haplotype associated with the first allele in the first individual.

28. The method of claim 19 further comprising determining a sequence variation which is associated with the first allele in the first individual.

29. The method of claim 28 wherein the sequence variation is a single nucleotide polymorphism (SNP).

30. The method of claim 28 wherein the sequence variation is an insertion.

31. The method of claim 28 wherein the sequence variation is a deletion.

32. A method of measuring allelic expression variation in a non-imprinted gene in a first individual, comprising:
reverse transcribing and amplifying mRNA from an individual heterozygous for a single nucleotide polymorphism (SNP) in a non-imprinted gene to form first cDNA from a first allele and second cDNA from a second allele;
hybridizing primers to first cDNA and second cDNA and differentially labeling those primers hybridized to first cDNA and second cDNA using fluorescent dye terminators and a single base extension reaction to form differentially labeled first and second primers;

33. comparing amount of differentially labeled first primers to amount of differentially labeled second primers using capillary electrophoresis, wherein a statistically significant difference in the amount of labeled first primers from the amount of labeled second primers indicates that the first and second alleles are differentially expressed in the first individual.

34. A method of measuring allelic expression variation in a non-imprinted gene in a first individual, comprising:
determining level of expression of an allele of a gene in a first individual displaying a phenotype;
determining level of expression of the allele in a population of control individuals, said control individuals not displaying the phenotype;
comparing level of expression of the allele in the first individual to level of expression in the population of control individuals, wherein a statistically significant difference in the levels of expression indicates that the allele in the first individual may be associated with the phenotype.

35. A method of measuring allelic expression variation in a non-imprinted gene in a first individual, comprising:
determining level of expression of an allele of a gene in a first individual, wherein a level of expression of the gene has been associated with a phenotype;
comparing level of expression of the allele in the first individual to level of expression in a first or second population of control individuals, wherein the first population of control individuals have the phenotype and wherein the second population of control individuals do not have the phenotype, wherein a statistically significant difference in the levels of expression between the first individual and the second population indicates that the first individual has the phenotype and wherein no statistically significant difference in the levels of expession between the first individual and the first population indicates that the first individual does not have the phenotype.
